(19) United States
(12) Patent Application Publication
(10) Pub. No.: US 2011/0253605 A1
(43) Pub. Date: Oct. 20, 2011

(54) ALGAE FILTRATION SYSTEMS AND METHODS

(75) Inventors: Qiang HU, Chandler, AZ (US); Milton SOMMERFELD, Chandler, AZ (US); Xuezhi ZHANG, Chandler, AZ (US); Aniket KALE, Chandler, AZ (US)

(73) Assignee: Arizona Board of Regents for and on Behalf of Arizona State University, Scottsdale, AZ (US)

(21) Appl. No.: 13/149,524

(22) Filed: May 31, 2011

Related U.S. Application Data
(63) Continuation of application No. PCT/US2011/028027, filed on Mar. 11, 2011.
(60) Provisional application No. 61/315,602, filed on Mar. 19, 2010.

(51) Int. Cl. B01D 29/075; B01D 29/76; B01D 29/64; B01D 29/66; A01H 13/00; B01D 29/23

(52) U.S. Cl. 210/90; 210/416.1; 210/413; 210/414; 210/411; 210/321.69

(57) ABSTRACT

Systems and methods for filtering algae from fluid including a piston and pressurized air system to scrape and clean algae from the filter.

This application is a continuation of and claims priority to PCT Application No. PCT/US2011/028027, filed Mar. 11, 2011 and entitled “ALGAE FILTRATION SYSTEMS AND METHODS,” and U.S. Provisional Patent Application Ser. No. 61/315,602 filed Mar. 19, 2010 and entitled “ALGAE FILTRATION SYSTEMS AND METHODS”, both of which are incorporated herein by reference in their entirety.

### BACKGROUND

A. Field of the Invention

Embodiments of the present invention relate generally to systems and methods for filtering algae from fluid. In particular, embodiments of the present invention concern the use of filtration systems and methods with a piston that can be used to scrape algae from the filter material.

B. Description of Related Art

Production of biofuel from algae is a very promising technology. Among alternative energy sources, algae represent a renewable biomass resource that is ready to be implemented on a large scale without any environmental or economic penalty. Due to CO₂ fixation by the algae, all the organic matter biodegraded is converted into biomass under photosynthetically oxygenated treatments. The photosynthetic efficiency of aquatic biomass is much higher (6-8%, on average) than that of terrestrial plants (1.8-2.2%, on average). Also, aquatic algae are readily adaptable to growing in different conditions, including fresh- or marine-waters.

Algae can be harvested by coagulation, flocculation, flotation, centrifugation, screen or membrane filtration, and gravity sedimentation. Unfortunately, none of the common industrial approaches have been proven to be economical and suitable for large-scale microalgae separation or removal. Recovery of biomass can be a significant problem because of the small size (3-30 μm diameter) of the algal cells and the large volumes or water that must be processed to recover the algae.

Screens or membrane filter are generally high efficient. However, the use of water jets to dislodge the algae from the screen or membrane can cause severe dilution of the harvested algae. Therefore, a cost-effective system and method of filtering algae from water and removing the algae from the screen or membrane filter is needed.

### SUMMARY

Embodiments of the present disclosure address issues related to systems and methods of filtering algae from water. In certain embodiments, the filtration system and method utilize a piston configured, water or pressurized air to scrape, scour and collect the filtered algae from the filter.

Typical algae culture concentration at the end of growth cycle and product accumulation phases is between 1-10 g/L. It is therefore desirable to filter the algae from the fluid utilizing systems and methods as disclosed herein.

Exemplary embodiments of the filtration systems disclosed herein can comprise a tubular metal mesh or a screen to support a filter. In certain embodiments, the metal is resistant to corrosion based on the components of the culture, and the filter cloth can be attached firmly to the metal. In exemplary embodiments, the pore size of the filter is in the range of micrometers and the material of the filter is smooth so that algae cake layer can be easily scraped or removed easily by the piston, water or air.

Embodiments of the filtration system comprise two fluid pathways: the permeate path through the filter and the retentate path, which is a flow through path in the filter and has a valve at the end called the retentate valve. Initially, the retentate valve is closed to operate the system in a dead end filtration mode. Algae-containing water enters the apparatus and algae will be retained on the filter. During the filtration process, the flow and pressure before and after the filter can be monitored. The culture accumulates in the filter and algae is concentrated and forms a cake on the filter surface as the water and the nutrients flow through the permeate pathway due to an increase in the pressure. The permeate flux drops as the process continues. When the tubular filter is filled with algae or the algae cake resistance is too high to obtain reasonable flux, the feed valve can be closed and the collection program is initiated.

Embodiments of exemplary filtration methods comprise: 1) draining the concentrated algae suspension inside the filter housing back to the algae container (2) using a piston to push the algae collected on the filter to an algae container; 3) backwashing the filter using water directed by pressurized air or pressurized air from the permeate side to dislodge remaining algae material from the filter; 4) backwashing the feed side of the membrane with air.

Exemplary embodiments can comprise a piston valve connected to the top of the tubular filter during filtration. A collection or retentate valve at the bottom of the filter can be opened and the scraping device moved through the filter to push the algae cake though the filter. Upon complete collection of the concentrated algae, the scraping device can be pulled back and returned to its original position.

After scraping, there may be algae particles remaining in the filter. These can be cleaned using a backwash. By increasing the pressure on the downstream of the permeate side of the system, the blocked particles on the surface of the filter are dislodged. In addition, air can be used to scour the algae particles off the filter surface into algae container.

It is contemplated that any embodiment discussed in this specification can be implemented with respect to any method or system of the invention, and vice versa. Furthermore, systems of the invention can be used to achieve methods of the invention.

The term “conduit” or any variation thereof, when used in the claims and/or specification, includes any structure through which a fluid may be conveyed. Non-limiting examples of conduit include pipes, tubing, channels, or other enclosed structures.

The term “reservoir” or any variation thereof, when used in the claims and/or specification, includes any body structure capable of retaining fluid. Non-limiting examples of reservoirs include ponds, tanks, lakes, tubs, or other similar structures.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The terms “inhibiting” or “reducing” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE FIGURES

FIG. 1 is a schematic side view of an exemplary embodiment of filtration system according to the present disclosure.

FIG. 2 is a schematic top view of components of the exemplary embodiment of FIG. 91.

### DESCRIPTION OF EXEMPLARY EMBODIMENTS

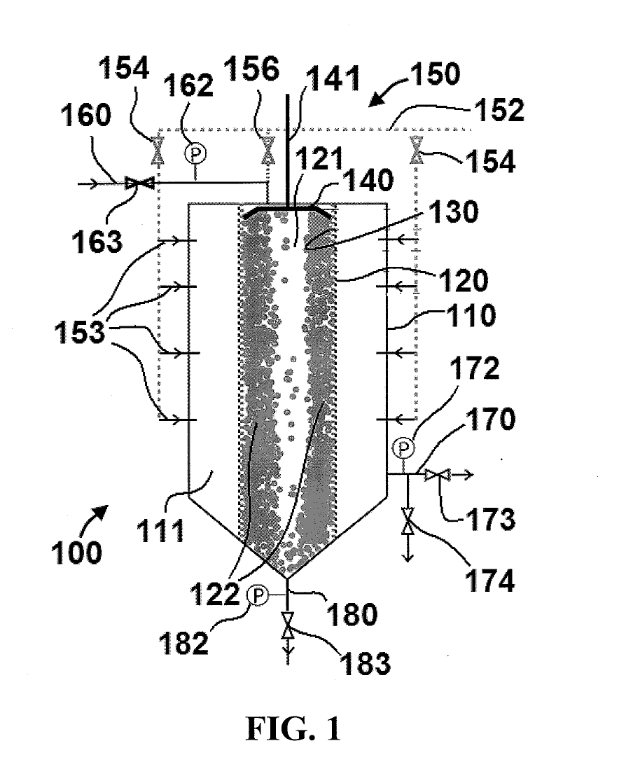

FIG. 1 is a schematic view of an exemplary embodiment of a filtration system 100 comprising a filter housing 110, a filter support 120 and a filter material 130. In this embodiment, filter housing 110 is constructed from stainless steel or polyvinylchloride (PVC) and is approximately 0.45 meters in diameter. In the exemplary embodiment shown, filter support 120 comprises a stainless steel or PVC tubular meshes or screen approximately 0.2 meters in diameter, with a nominal pore size of 50 microns. In this embodiment, filter material 130 comprises a stainless screen, cellulose acetate (CA), polysulfone (PS), polyethylene (PE), polyethersulfone (PES), polyvinylidene difluoride (PVDF) or PVC membrane with a nominal pore size of less than 1 microns. In addition, filtration system 100 comprises a piston 140 extending into one end of filter material 130. As explained in more detail below, piston 140 may be used to remove filtered material from filter material 130.

Filtration system 100 further comprises a backflow system 150 configured to direct air or permeate across filter material 130 in a direction that is reverse to the direction of flow across filter material 130 during normal operation. Backflow system 150 comprises conduit 152 (e.g., tubing or piping) configured to direct air into filter housing 110.

Filtration system 100 comprises an inlet conduit 160 configured to allow algae-containing fluid to enter an inner volume 121 of filter support 120 and filter material 130 during operation. Inlet conduit 160 can also comprise a pressure indicator (e.g., a gauge) 162 that monitors the fluid pressure prior to the fluid entering inner volume 121.

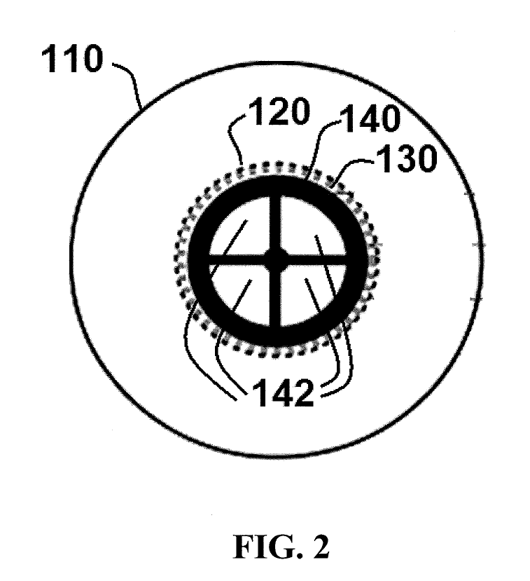

As shown in the top schematic view of FIG. 2, piston 140 comprises apertures 142 configured to allow the algae-containing fluid to pass through the central portion of piston 140. During operation, the fluid passes from inner volume 121 through filter material 130 and filter support 120 and into an outer volume 111 between filter support 120 and filter housing 110. As the fluid passes through filter material 130, algae 122 is separated from the fluid and remains in inner volume 121.

The fluid can exit filter housing 110 via an outlet conduit 170 and be sent for further processing or recycling. Outlet conduit 170 can also comprise a pressure indicator (e.g., a gauge) 172 that monitors the fluid pressure downstream of filter housing 110.

During operation, the pressure at pressure indicators 162 and 172 can be monitored to determine the pressure across filter material 130. When the differential pressure reaches a predetermined value (e.g., 15 psig), the user may cease flow of the fluid through filter material 130 by closing an inlet valve 163 and outlet valve 173. In other embodiments, the flow of fluid may be stopped at predetermined time intervals, even if the differential pressure remains below the pre-determined value. A drain valve 174 can then be opened to drain water back to a supply tank.

A collection conduit 180 (comprising a collection valve 183 and a pressure indicator (e.g., a gauge) 182 can then be opened to collect the harvested algae. During harvesting, piston 140 is pushed downward from the position shown in FIG. 1 towards collection conduit 180. As piston 140 is pushed downward, it scrapes algae 122 from filter material 130. Algae 122 can then be forced out through collection conduit 180.

After algae 122 has been collected or harvested, filter material 130 can be cleaned by backflow system 150. In this embodiment, backflow system 150 comprises valves 154 and nozzles 153. During the cleaning process, valves 154 can be opened to allow higher pressure air (or other suitable cleaning fluid) to enter outer volume 111 between filter housing 110 and filter support 120. The introduction of higher pressure air into outer volume 111 can create a pressure differential across filter material 130 and dislodge algae 122 from filter material 130. The dislodged algae 122 can then be pushed down to the bottom of filter housing 110 by pressurized air via valve 156 and be collected via collection conduit 180. With collection valve 183 open, algae 122 can be directed to a collection vessel. After algae 122 is collected, collection valve 183 can be closed and the system prepared for additional filtration. For example, piston 140 can be returned to the position shown in FIG. 1, drain valve 174 can be closed, and outlet valve 173 and inlet valve 163 can be opened to allow water to pass through filtration system 100 as previously described.

In certain exemplary embodiments, the clearance between piston 140 and filter material 130 is between 0.1 and 1.0 mm. In specific embodiments, piston 140 may be constructed from rubber and be coupled to a stainless steel support rod 141.

In certain embodiments, piston 140 may comprise a retractable scraper constructed from polypropylene or stainless steel that can be adjusted to increase or decrease the outer diameter of piston 140. Such a configuration can allow for variation in the diameter of filter material 130.

In still other embodiments, piston 140 may comprise a nylon brush that engages filter material 130. Such a configuration may be useful when the algae layer on filter material 130 is thinner than the clearance between rubber portion of piston 140 and the inner diameter of filter material 130.

### REFERENCES

The following references are herein incorporated by reference in their entirety.
U.S. Pat. No. 3,951,805
U.S. Pat. No. 3,983,036
U.S. Pat. No. 4,255,261
U.S. Pat. No. 4,465,600
U.S. Pat. No. 4,869,823
U.S. Pat. No. 4,554,390
U.S. Pat. No. 5,562,251
U.S. Pat. No. 5,254,250
U.S. Pat. No. 6,063,298
Borowitzka, M. A. (1999). Commercial production of microalgae: ponds, tanks, tubes, and fermenters. J Biotechnol 70, 313-321.
Chisti, Y. (2007). Biodiesel from microalgae. Biotechnol Adv 25, 294-306.
Daigger, G. T, B. E. Rittmann, S. S. Adham, and G. Andreottola (2005). Are membrane bioreactors ready for widespread application? Environ. Sci. Technol. 39: 399A-406A.
Rittmann, B. E. (2008). Opportunities for renewable bioenergy using microorganisms. Biotechnol. Bioengr. 100: 203-212.
Rittmann, B. E. and P. L. McCarty (2001). Environmental Biotechnology: Principles and Applications. McGraw-Hill Book Co., New York.

1. A separation system comprising:
a filter housing;
a filter support disposed within the filter housing, wherein the filter support is tubular and comprises a first end and a second end;
an outer volume between the filter support and the filter housing;
an inner volume within the filter support;
a filter material coupled to the filter support, wherein the filter material is disposed within the inner volume and wherein the filter material comprises a feed side
and a permeate side;
a piston disposed within the inner volume;
an inlet conduit, wherein the inlet conduit is configured to direct a mixture of water
and algae to the inner volume;
water and or air on the permeate side;
air on the feed side; and
an outlet conduit, wherein the outlet conduit is coupled to the outer volume.

2. The separation system of claim 1 wherein the filter material filters and harvests the algae from the water during operation.

3. The separation system of claim 1 wherein the piston is configured to move from the first end of the filter support to the second end of the filter support.

4. The separation system of claim 3 wherein the piston is configured to scrape algae from the filter material as the piston moves from the first end of the filter support to the second end of the filter support.

5. The separation system of claim 1 further comprising a collection conduit coupled to the inner volume, wherein the collection conduit is configured to collect algae that have been scraped from the filter material by the piston.

6. The separation system of claim 1 further comprising a backflow system configured to direct a fluid from the outer volume to the inner volume.

7. The separation system of claim 6 wherein the fluid is air directed to dislodge algae from the filter material.

8. The separation system of claim 1 wherein air can be used to scour the algae off the filter surface and push it into the algae container.

9. The filtration system of claim 1 wherein the piston comprises a retractable scraper configured to increase or decrease the outer diameter of the piston.

10. The separation system of claim 1 wherein the piston comprises a nylon brush that engages the filter material.

11. The separation system of claim 1 wherein the piston comprises one or more apertures configured to allow fluid to pass through a central portion of the piston.

12. The separation system of claim 1 wherein the filter material comprises a screen or membrane filter or membrane with a nominal pore size of less than 1 micron.

13. The separation system of claim 1 wherein the filter support comprises a nominal pore size of 50 microns.

14. The separation system of claim 1 wherein the filter support comprises a tubular mesh or screen approximately 0.2 meters in diameter.

15. The separation system of claim 1 wherein the inlet conduit comprises a first pressure indicator and the outlet conduit comprises a second pressure indicator.
